(19) United States
(12) Patent Application Publication
(10) Pub. No.: US 2021/0181215 A1
(43) Pub. Date: Jun. 17, 2021

(54) TREATMENT AND DIAGNOSIS OF ANAEMIA

(71) Applicant: THE UNIVERSITY COURT OF THE UNIVERSITY OF ABERDEEN, Aberdeenshire (GB)

(72) Inventors: Mark VICKERS, Aberdeenshire (GB); Robert BARKER, Aberdeenshire (GB); Huan CAO, Aberdeenshire (GB)

(21) Appl. No.: 16/760,556

(22) PCT Filed: Oct. 31, 2018
(86) PCT No.: PCT/EP2018/079822

§ 371 Date: Apr. 30, 2020

(30) Foreign Application Priority Data
Oct. 31, 2017 (GB) 1717977.1

(51) Int. Cl. G01N 33/90; A61K 31/722; G01N 33/50; C12N 15/113

(52) U.S. Cl. CPC G01N 33/90; A61K 31/722; G01N 33/502; G01N 2800/22; G01N 2333/70596; G01N 2400/38; G01N 2400/22; C12N 15/1138

(57) ABSTRACT

The present invention relates to compounds and compositions that can be used in the treatment and diagnosis of anaemias, particularly haemolytic anaemias such as sickle cell anaemia. Methods of selecting such compounds and compositions are also provided.

### TECHNICAL FIELD

The present invention relates to compounds and compositions that can be used in the treatment and diagnosis of anaemias, particularly haemolytic anaemias such as sickle cell anaemia.

### BACKGROUND

The health of cells is continually monitored and damaged or old cells removed in a process termed efferocytosis. Phagocytic cells, mainly macrophages, make this decision on the basis of the relative strengths of ‘eat me’ versus ‘don't eat me’ signals displayed on cellular surfaces. The main mammalian endogenous (self) ‘eat me’ ligand is considered to be phosphatidylserine (PS) (Fadok, V. A. 1992), a phospholipid usually confined to the inner leaflet of plasma membranes by the operation of an active process catalysed by flippases. Cell death by either apoptosis or necrosis results in a redistribution of PS to the outer leaflet, where it can be recognised by receptors expressed on the surface of phagocytic cells, such as macrophages. Several receptors for PS have been described, both receptors that bind directly, such as TIM-4, BAI1 and Stabilin 2, or indirectly, recognising bridging molecules, such as GAS6. Other ‘eat me’ signals have also been identified, such as calreticulin recognised by LRP1 (Gardai, S. J. 2005) and thrombospondin recognised by the vitronectin receptor (Freeman SA, Grinstein S, 2014).

Phagocytic cells also eat microbes and most microbe-associated ‘eat me’ signals are glycans associated with cell walls. For instance, high mannose structures are found on the surface of many procaryotes and fungi, and are mainly recognised by macrophages via C-type lectins (a class of innate immune receptors). Glycans have also been implicated in the efferocytosis of mammalian cells (Duvall, E. 1985; Falasca, L. 1996; Dini, L. 2002; Gardai, S. J. 2006; Bil, R. O. 2012), but the structural basis for this role has not been characterised.

In humans, the cell type with highest turnover is the red blood cell (RBC; erythrocyte). Approximately 2-3×10¹¹ RBC are phagocytosed daily by specialised macrophages lining splenic and hepatic sinusoids. The signals mediating uptake of effete red cells have been thought to comprise a mixture of PS exposure, loss of sialic acids, auto-antibody binding with fixation of complement, and loss of CD47 expression ( ). However, the evidence underlying these mechanisms has significant flaws. In order to identify glycans that might act as novel ‘eat me’ signals to stimulate uptake by macrophages, the inventors have used unbiased analytical techniques to perform systematic analyses of the glycome of red blood cells (RBC; red cells; erythrocytes), the conclusions from which are described below.

Many anaemias are characterised by short red cell lifespans, mediated by accelerated by high levels of RBC clearance. One of the commonest and severest of these is the genetically determined condition sickle cell anaemia, which is characterised by extensive morbidity and a haemolytic anaemia, with red cell lifespans typically about 10 rather than the usual 120 days. About two thirds of haemolysis in SCD is extravascular, mediated through phagocytosis by splenic and hepatic macrophages. The remaining third results from lysis of red cells within the vascular space. Haemolysis has been presumed to result from either exposure of phosphatidylserine or abnormal rheology, but the evidence for these beliefs has significant flaws.

### SUMMARY OF THE INVENTION

The inventors have identified the surprising importance of mannose motifs in the detection and disposal of damaged RBC by phagocytic cells. Discovery of this mechanism raises the possibility that it might also be important in the pathogenesis of diseases that involve haemolysis. The commonest such disease is sickle cell anaemia. The inventors investigated this possibility and showed that RBC from patients with sickle cell disease express very high surface levels of mannose, which are indeed also recognised by innate immune receptors expressed by phagocytic macrophages. The present disclosure describes methods to detect the presence of mannose residues exposed on RBC from patients with sickle cell disease (SCD) so that they can be differentiated from healthy RBC. Further, it is shown that the destruction of damaged or diseased RBC can be inhibited by using agents that inhibit the interaction of exposed mannose residues with clearance mechanisms, including phagocytic receptors.

Moreover, the inventors have found that high mannose is exteriorised by other types of damaged cells, including neutrophils and neuron derived cells (neuroblastoma). This finding opens a new therapeutic approach in diseases such as neurodegenerative diseases, where the phagocytosis of damaged cells is undesirable.

Accordingly, at its broadest, the invention provides a novel therapeutic approach for diseases affecting cellular lifespan and clearance. The therapeutic approach involves targeting a previously undisclosed class of molecules, exteriorised high mannoses, and/or the innate immune receptors that bind the mannose residues.

Thus, in one aspect, the invention provides therapeutic agents for use in treating the human or animal body, wherein the therapeutic agent comprises an inhibitor of the interaction between an innate immune receptor and exteriorised high mannose expressed at the surface of cells. These therapeutic agents may be used in the treatment of diseases where the phagocytosis of damaged cells is undesirable, for instance neurodegenerative disease. The therapeutic agent is defined in more detailed herein in conjunction with the other aspects of this invention.

In a further aspect, the invention provides therapeutic agents for use in treating a haemolytic anaemia in a mammalian subject, the method comprising administering the therapeutic agent to the mammalian subject, wherein the therapeutic agent comprises an inhibitor of the interaction between an innate immune receptor and glycans expressed on the surfaces of diseased red blood cells.

In related aspects, the invention provides therapeutic agents for use in treating sickle cell disease in a mammalian subject, the method comprising administering the therapeutic agent to the mammalian subject, wherein the therapeutic agent comprises an inhibitor of the interaction between an innate immune receptor and glycans expressed on the surfaces of diseased red blood cells.

In some embodiments, the mammalian subject is a human. In some embodiments the haemolytic anaemia is sickle cell anaemia.

In some embodiments, the sickle cell disease is an acute sickle-cell crisis, for instance haemolytic crisis, vaso-occlusive crisis, splenic sequestration crisis, aplastic crisis or acute chest syndrome.

In most embodiments, the innate immune receptor is a C-type lectin. In some embodiments, the C-type lectin is the mannose receptor (CD206).

In some embodiments, the therapeutic agent is a decoy ligand that binds to the innate immune receptor. Preferably, the decoy ligand comprises an oligosaccharide or polysaccharide. The oligosaccharide or polysaccharide may comprises mannose or an analogue thereof. In some embodiments, the decoy ligand is mannan. In some embodiments, the decoy ligand comprises mannose congeners.

The decoy ligand may comprise an oligosaccharide or polysaccharide which comprises N-acetylglucosamine or an analogue thereof. In some embodiments, the decoy ligand is chitin.

In some embodiments, the therapeutic agent is an anti-CD206 antibody or fragment thereof. In other embodiments the therapeutic agent is a nucleic acid that causes CD206 knock-down. The nucleic acid may be an siRNA, a microRNA, a shRNA, or an analogue thereof.

In another aspect, the invention provides a glycoprotein comprising a human membrane skeletal polypeptide that is associated with one or more high mannose glycans. Preferably, the membrane skeletal polypeptide is a membrane skeleton protein, such as spectrin.

In a related aspect, the invention provides a molecule or antibody that specifically binds a glycoprotein comprising a human membrane skeletal polypeptide that is covalently linked to one or more high mannose glycans in such a way that it inhibits recognition by molecules of the innate immune system that mediate cellular clearance. The molecule or antibody may be provided for use in treating a haemolytic anaemia in a mammalian subject, the method comprising administering the therapeutic agent to the mammalian subject. The invention also provides methods for producing the antibodies disclosed herein, the methods comprising administering the glycoprotein described herein to a non-human mammal. The administration protocols, use of adjuvants, etc, form part of the common general knowledge and the skilled person understands that antibodies produced by these and other methods can be adapted, humanized, etc, as described herein and elsewhere.

In some embodiments, the therapeutic agent of the invention comprises a pharmaceutically acceptable excipient carrier, buffer and/or stabiliser.

In a further aspect, the invention provides a method of selecting a therapeutic agent for treating a haemolytic anaemia, the method comprising; (i) contacting an innate immune receptor with a candidate agent; (ii) measuring the binding affinity of the immune receptor for the candidate agent; and (iii) selecting the candidate agent as a therapeutic agent if the binding affinity is above a predetermined threshold. In related aspects, the invention provides methods of selecting a therapeutic agent for treating sickle cell disease, the method comprising; (i) contacting an innate immune receptor with a candidate agent; (ii) measuring the binding affinity of the immune receptor for the candidate agent; and (iii) selecting the candidate agent as a therapeutic agent if the binding affinity is above a predetermined threshold. The innate immune receptor may be a C-type lectin, such as CD206 or CD209. In some embodiments, the selected therapeutic agent is then provided by formulating it a pharmaceutical composition. The pharmaceutical composition may be packaged together with instructions for administration.

In a further aspect, the invention provides a method of selecting a therapeutic agent for treating a haemolytic anaemia, the method comprising; (i) contacting a glycoprotein whose glycan interacts with an innate immune receptor mediating efferocytosis with a candidate agent; (ii) measuring the binding affinity of the glycoprotein for the candidate agent; and (iii) selecting the candidate agent as a therapeutic agent if the binding affinity is above a predetermined threshold. In related aspects, the invention provides methods of selecting a therapeutic agent for treating sickle cell disease, the method comprising; (i) contacting a glycoprotein with a candidate agent; (ii) measuring the binding affinity of the immune receptor for the candidate agent; and (iii) selecting the candidate agent as a therapeutic agent if the binding affinity is above a predetermined threshold. The glycoprotein may be a membrane skeletal protein, such as spectrin. In some embodiments, the selected therapeutic agent is then provided by formulating it a pharmaceutical composition. The pharmaceutical composition may be packaged together with instructions for administration.

In some embodiments the molecule is the extracellular portions of lectins that are able to bind high mannoses, which would act as a decoy receptor able to inhibit full length phagocytic receptors from binding.

In some embodiments, the therapeutic agent is a fragment of CD206 or mannose binding protein that binds mannose residues but lacks the ability to engage with mechanisms effecting efferocytosis

In some embodiments the molecule is the portion of a protein that binds high mannoses with high affinity, but which lacks the effector part of the molecule to cause cellular clearance. In a further embodiment, this is the mannose binding portion of mannose binding protein.

In other aspects, this invention relates to methods of providing diagnostically relevant information. This information is relevant to the diagnosis and monitoring of haemolytic anaemias and/or sickle cell diseases, and these methods comprise detecting the presence or absence of high mannose glycans on the surface of red blood cells in a sample that has been obtained from the subject. High mannose glycans that form part of glycoproteins described herein or exteriorised spectrin may be specifically detected.

### FIGURES

FIG. 1: High mannoses are found on red cell ghosts, especially oxidised and sickle cells. Glycomic mass spectrometric profile of N-linked glycans released from RBC ghosts, (A) healthy RBC, (B) oxidised RBC, (C) RBC from patient with SCD. High mannose structures are identified by charge/mass (m/z) ratio (1579: Man5GlcNAc2, 1783/4: Man6GlcNAc2, 1988: Man7GlcNAc2, 2192: Man8GlcNAc2, 2396: Man9GlcNAc2). The intensities of signal observed for each of these structures are summarised in (D). It can be seen that high mannoses are particularly prominent relative to sialylated glycans in ghosts from patients with SCD.

FIG. 2: High mannoses are expressed at high levels on the surface of oxidised RBC and very high levels on RBC from patients with SCD. (A): Surface binding of mannose binding lectin GNA detected by FACS. Such binding is specific for mannose binding as it is inhibitable by mannan and chitin (B) and correlates closely with another mannose specific lectin (NPL) (C). (D) shows binding of antibodies versus epitopes expressed on the cytoplasmic side of the plasma membrane are unchanged on these cells, indicating no non-specific loss of membrane integrity. (E) phase contrast fluoresecent microscopy shows that GNA binding appears as discrete patches on the plasma membrane and that similar structures are can be detected in an intracellular location in permeabilised healthy cells.

FIG. 3: High mannoses are associated with the membrane skeletal protein spectrin. (A) Lectin blot of RBC ghosts electrophoresed on SDS-PAGE, indicating a single major band running at 260 kD, corresponding to alpha-spectrin, as seen on Coomassie staining. The signal can be degraded by prior incubation with PNGase F or endo H indicating the N-linked nature of the signal. A similar band is also detected by NPL. When NPL is used to immunoprecipitate from RBC ghosts before Western blotting, a single band can be detected by an anti-spectrin antibody. This signal is also degradable by prior incubation with PNGase F and no band is detected when Mal II is used as the precipitating lectin. (B): fluorescent super-resolution microscopy shows GNL binding occurs in patches (yellow) coincident wirth the membrane skeleton stained using anti-spectrin (blue). (C) polyclonal anti-spectrin binds the surface of RBC from patients with SCD, in contrast to other membrane proteins shown in FIG. 2B.

FIG. 4: Mannose mediate uptake of damaged/diseased Red Blood Cells. (A): Example of uptake of RBC by human monocyte derived macrophages. Macrophage cytoplasm is labelled green using anti-mannose receptor antibody, nuclei are labelled blue with DAPI and RBC are labelled red. (B): RBC subject to oxidative damage are taken up at greater rates than undamaged and this difference is abrogated by incubation with mannan, chitin, anti-CD206 antibody or knock-down of expression of CD206 by siRNA, all of which disrupt high mannose-mannose receptor interactions. Phagocytosis of control latex beads is unaffected by these blocking agents. (C) RBC from patients with SCD containing HbSS are also taken up by macrophages with high efficiency relative to healthy RBC containing HbAA and uptake is similarly blocked by mannan, chitin or blocking anti-CD206 antibody.

FIG. 5: Mannose exposure correlates with markers of RBC turnover. (A) Scatterplot showing log transformed mannose exposure of RBC, measured by binding of fluorescently labelled GNA lectin, is indicated on the horizontal axis, with three markers of red cell turnover marked on the vertical axes as labelled. Peripheral blood samples were sampled from several groups: healthy volunteers, random samples from patients with normal and high HbA1c levels, patients homozygous for Hb S and RBC indices indicating no concomitant β-thalassaemia, patients homozygous for Hb S and RBC indices indicating concomitant β-thalassaemia, compound heterozygotes for Hb S and β-thalassaemia, compound heterozygotes for HbS and HbC, compound heterozygotes for HbS and haemoglobin D Punjab, patients homozygous for HbSS and taking therapeutic hydroxycarbamide.

FIG. 6: Factors ameliorating sickle cell disease severity are associated with less mannose exposure. (A) Same data as FIG. 5, but horizontal axis indicates source of samples. (B) FACS plot indicating the assay is able to distinguish RBC with HbAA versus HbAS well enough to enable estimation of the proportion of transfused cells in a patient before and after a blood transfusion.

FIG. 7: The mannose exposure pathway operates in non-erythroid cells. SHSy5y neuronal cells were oxidised (30 minutes) by copper sulphate and ascorbic acid as for erythrocytes and compared to non-oxidised cells. Immuno-fluorescence images of intracellular SPTBN1 staining (green) is shown with DAPI (blue) in healthy SHSy5y cells. Surface staining of SPTBN1 (green) is merged with bright field for healthy non-oxidised SHSy5y neuronal cells (top left) and oxidised SHSy5y cells (bottom left). Higher magnification of oxidised SHSy5y cells is shown as merged image (top right) and SPTBN1 (green) staining only (bottom right).

FIG. 8: Quantification of erythrocyte clearance by the two-toned efferocytosis assay. Cell Trace Far Red (CTFR, red) stained oxidised erythrocytes were added to HMDM for 3 hours, washed with PBS and stained with GPA-FITC antibody (Green) prior to immunofluorescence microscopy: GPA-FITC only (i), GPA-FITC and CTFR (ii), bright field only (iii) and merged (iv). CTFR (red) single positive cells are counted as having been efferocytosed (letter E). Double positive (CTFR and GPA) cells are not counted as having been efferocytosed but as bound to the macrophage cell surface. 50 μm scale bar as shown.

### DETAILED DESCRIPTION

As noted herein, the disposal of unwanted or dying cells is a key biological process driven by the display of ‘eat me’ signals that are recognised by phagocytes. Dead cells are removed by phagocytes, mainly macrophages, by a process termed ‘efferocytosis’. Phosphatidylserine (PS) has received most attention as a phagocytic marker of dying cells, but it is widely accepted that other important signals for efferocytosis remain to be identified [10]. In particular, recognition of glycans on the surface of dying cells has been implicated in clearance [3], [5], [6], [9], [11], but the structural basis for this role has not previously been understood.

The present disclosure shows that the mannose displayed on oxidised RBC represented a ‘eat-me’ signal, which was previously unknown in the context of human cells. The inventors disclose a novel mechanism whereby high mannose structures, which are normally unavailable for extracellular inspection, become visible to inspecting macrophages and so stimulate uptake by phagocytic cells. Cellular damage causes specific high mannoses to become presented in discrete patches at the cell surface. The inventors confirmed the importance of this pathway to human pathology by demonstrating that sickle cell disease (SCD) is characterised by prominent exposure of mannose in patches on the surface of many RBC, that the severity of haemolysis correlates with the quantity of exposed mannoses and by demonstrating that blocking the recognition of mannose inhibits the accelerated uptake of sickle RBC by macrophages.

The high mannose structures are associated with spectrin, a membrane skeletal protein that has important roles in maintaining membrane integrity and cellular shape and is located just under the plasma membrane. Spectrin is the main protein that determines the shape of the cell and is also involved in the organisation of specialised membrane domains [22]. Non-erythroid isoforms of spectrin are also ubiquitous in nucleated cells [22].

Binding of mannosylated spectrin exposed at the cell surface would enable phagocytic cells to bind a rigid membrane skeletal structure that encloses the cell, to thus effect efferocytosis.

The inventors found that at least one immune receptors; CD206 (‘the mannose receptor’) is important in erythrocyte uptake by the use of blocking antibody and siRNA knock-down. These findings, and other features that illustrate the invention, are discussed further below.

Abbreviations

ACD Acid Citrate Dextrose

CR Cysteine Rich region

CRD Carbohydrate Recognition Domain

CTFR Cell Trace Far Red

DAMP Damage Associated Molecular Pattern

FITC Fluorescein Isothiocyanate

GNA Galanthus nivalis Lectin

GPA Glycophorin A

HMDM Human Monocyte Derived Macrophages (may also be referred to as MDMΦ)

NPL Narcissus pseudonarcissus Lectin

O-GlcNAc O-linked N-acetylglucosamine

PBS Phosphate Buffered Saline

PFA Paraformaldehyde

RBC Red Blood Cells

SIM Structured Illumination Microscopy

SCD Sickle Cell Disease

TEM Transmission Electron Microscopy

Pharmaceutical Compositions

Therapeutic agents according to the present disclosure are preferably provided as pharmaceutical compositions. Pharmaceutical compositions according to the present disclosure, and for use in accordance with the present disclosure, may comprise, in addition to the active ingredient, e.g. an inhibitor of innate immune receptor binding to glycosylated membrane skeletal polypeptides, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, the active ingredient will be in the form of a parenterally acceptable aqueous solution which is pyrogen-free and has suitable pH, isotonicity and stability. Formulations suitable for parenteral administration (e.g., by injection), include aqueous or non-aqueous, isotonic, pyrogen-free, sterile liquids (e.g., solutions, suspensions), in which the therapeutic agent is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage

It will be appreciated by one of skill in the art that appropriate dosages of the therapeutic agent and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In general, a suitable dose of the therapeutic agent is in the range of about 100 ng to about 25 mg (more typically about 1 μg to about 10 mg) per kilogram body weight of the subject per day. Flat dosing may also be considered (.ie. not dependent on body weight or body surface area). Where the active compound is a salt, an ester, an amide, a prodrug, or the like, the amount administered is calculated on the basis of the parent compound and so the actual weight to be used is increased proportionately.

Duration of Treatment

A treatment regimen based may preferably extend over a sustained period of time. The particular duration would be at the discretion of the physician. For example, the duration of treatment may be at least 1, 2, 3, 4, 5, 6, 7, 8, 9, 10, 11, 12 months, or longer, at least 2, 3, 4, 5 years, or longer. In some embodiments, the duration of treatment will be between 6 and 12 months. In some embodiments, the duration of treatment will be between 1 and 5 years.

Decoy Receptors

In some embodiments, the therapeutic agent of the invention is not a direct inhibitor of the innate immune receptors discussed herein, but is instead a decoy receptor. The skilled person will understand that a decoy receptor of an innate immune receptor means a soluble (or solubilised) receptor that binds to the same or similar ligands as the innate immune receptor. For instance, the decoy receptor of the invention may be a solublised C-type lectin, or the (soluble) CRD of a C-type lectin. For instance, the decoy receptor may be solubilised CD206 or solubilised CD209, or the soluble CRD of CD209 or CD206. One or more amino acid mutations may be present in the decoy receptor, which were not present in the wild type receptor (i.e. deletions, insertions and/or substitutions). The skilled person will understand that receptors such as CD206 and CD209 can be solubilised e.g. by fusing the ligand binding domain of the receptor with the Fc domain of a human monoclonal antibody. Hence, in some embodiments of these aspects of the invention, the therapeutic agent is a fusion protein comprising the Fc domain of a human monoclonal antibody fused to the CRD of CD206 or fused to the CRD of CD209.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Journal of Immunology 170:4854-4861).

Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, Glm2, G1m3, non-G1m1 [that, is any allotype other than Glm1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulin sequences can be derived from any species, including human, murine, or rabbit origin.

“Antibody fragments” comprise a portion of a full-length antibody, generally the antigen binding or variable region thereof. Examples of antibody fragments include Fab, Fab′, F(ab)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly homogeneous, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact human antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Pharmaceutical Compositions and Their Use in Medicine

The pharmaceutical compositions and formulations described herein are useful, for example, in methods of treatment of a disorder as described herein.

Use in Methods of Therapy

Another aspect of the present invention pertains to a pharmaceutical composition or formulation, as described herein, for use in a method of treatment of the human or animal body by therapy, for example, for use a method of treatment of a disorder as described herein.

Use in the Manufacture of Medicaments

Another aspect of the present invention pertains to use of a pharmaceutical composition, as described herein, in the manufacture of a pharmaceutical formulation, as described herein, for the treatment of a disorder (e.g., haemolytic anaemia or a sickle cell disease), as described herein.

In one embodiment, the medicament comprises the therapeutic agent as described herein.

Methods of Treatment

Another aspect of the present invention pertains to a method of treatment, for example, of a disorder (e.g., haemolytic anaemia or a sickle cell disease) as described herein, comprising administering to a patient in need of treatment a therapeutically effective amount of a pharmaceutical composition or formulation, as described herein.

The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human being. Furthermore, the subject/patient may be any of its forms of development, for example, a foetus. In one preferred embodiment, the subject/patient is a human being.

Spectrin

Spectrin is a major component of the membrane skeleton. The inventors found that one membrane skeletal protein, spectrin, is associated with high mannose species in RBC and also in other cell types.

Efferocytosis

Efferocytosis is the process by which dead and dying cells are taken up by phagocytic cells. Like phagocytosis, efferocytosis is initiated by receptors engaging with their cognate ligand over an extended surface area of the phagocytic cell plasma membrane. This initiates a complex process, involving over 200 proteins, resulting in a membrane skeletal driven reordering of the cell membrane to engulf the target cell. An important concept underlying this proposal is that it is the bringing together of homologous plasma membrane receptors together into clusters that cause co-operative enzymatic activity from their intracytoplasmic and transmembrane domains and subsequent triggering of effero/phagocytosis. This process is not dependent on any functional property of the external ligand binding domains beyond ligand recognition.

High Mannose

High mannoses were previously thought to be more typically encountered on the surfaces of procaryotes or fungi and are recognised by phagocytic cells such as macrophages, mainly using C-type lectin receptors.

Red Blood Cells (RBC)

Red blood cells (RBC) were chosen as the main targets for macrophage uptake in this study. They are amongst the most abundant cell types in the human body and their turnover represents a major physiological process that is of high clinical relevance, with many inherited and acquired diseases directly affecting RBC lifespan and clearance. The inventors used human RBC oxidatively stressed by copper sulphate, which results in uptake by human monocyte-derived macrophages (HMDM), as a surrogate for aging.

Sickle Cell Disease (SCD)

Sickle cell disease is now the most common single gene disease in the world, affecting 20-25 million people globally and causing considerable morbidity and mortality.

Postulating that this novel phagocytic uptake pathway might be important in mediating pathological haemolysis as well as physiological red blood cell turnover, the inventors examined whether this pathway might be important in sickle cell disease (SCD), which is characterised by a poorly understood chronic haemolytic state. The inventors documented remarkably high surface expression of high mannose containing patches and confirmed that sickle cells are taken up at by cultured macrophages in vitro at rates much higher than healthy red cells. This uptake is blocked by congeners of mannoses and blocking antibodies to CD206.

Identification of Exteriorised High Mannose Structures

The inventors performed an unbiased glycomic survey of human red blood cell membranes identified novel N-linked high mannose structures, which are sequestered inside healthy cells on spectrin, the major protein of the internal membrane skeleton, but exteriorised when dying, as a dominant signal for uptake by macrophages.

A panel of lectin probes was used to demonstrate that the mannose species were available as discrete patches on the surface of RBC that had been stressed by oxidation and cells from patients with SCD, but not detectable on untreated, healthy cells. Proteomic analyses revealed that the N-linked high mannose structures decorated spectrin, the major component of the membrane skeleton, consistent with the intracellular location in healthy cells. Super resolution microscopy visualised co-localisation of mannose with spectrin in membrane protrusions of oxidised RBC. The decoration of spectrin with N-linked mannose, and exteriorisation on effete cells, are shared with nucleated cells, since similar phenomena were also observed in a neuronal cell line.

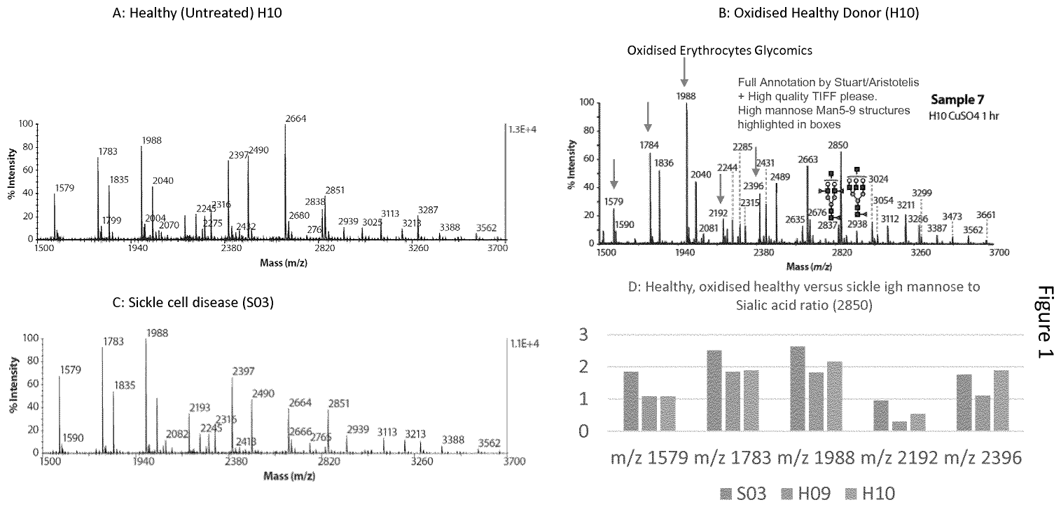

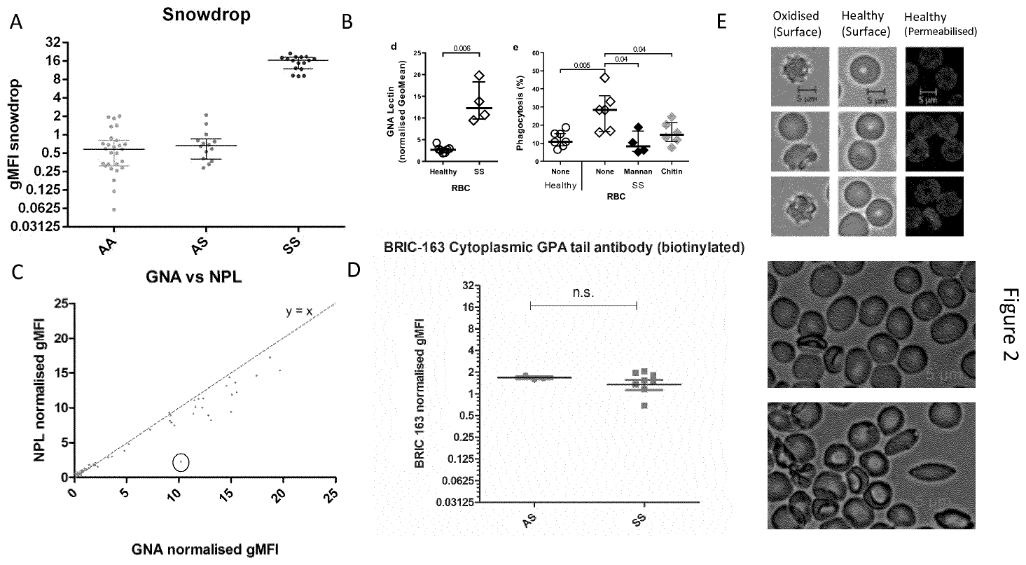

The inventors first exploited unbiased analytical techniques that now enable a more systematic characterisation of the glycome of cells. N- and O-linked glycans were purified from plasma membranes (ghosts) of freshly prepared untreated RBC and oxidatively damaged cells. Analyses by mass spectroscopy of the N-linked structures from both untreated and oxidised RBC identified abundant high mannose species, including Man5GlcNAc2, Man6GlcNAc2, Man7GlcNAc2, Man8GlcNAc2 and Man9GlcNAc2in addition to the expected series of complex N-glycans (FIG. 1). The presence of N-linked high mannose species on the plasma membrane was noteworthy since, in the cells of higher eukaryotes, such structures are generally considered to represent intermediates in the synthesis of complex glycans, a pathway which would be absent in mature RBC. High mannose species have not previously been considered to be ‘eat-me’ ligands in efferocytosis, but this new possibility required to be tested, since they are frequently found in microbial cell walls [7], where they can stimulate phagocytosis [19].

Implications of the Present Findings

This disclosure establishes the principle of using cellular surface mannose expression to detect and measure damaged or diseased versus healthy cells. Thus the proportion of RBC containing sickle cell haemoglobin can be readily measured. In addition, the degree of damage can be measured, which may be useful for titration of treatments of haemolytic anaemias.

This disclosure also establishes the principle of inhibiting the novel phagocytic uptake pathway as a method of controlling the haemolysis of sickle cell disease. In light of these findings, the skilled person will appreciate that inhibitors of the immune receptors described herein can be used as clinical interventions. For instance, receptor-blocking antibodies, decoy ligands comprising truncated high mannose, such as mannan or chitin or analalogues, or small molecules will find utility.

Moreover, the inventors expect other haemolytic anaemias will be mediated by this mechanism and will be amenable to this approach, as will other diseases of the red blood cells, such as glucose-6-phosphate dehydrogenase deficiency. The inventors have also shown that this mechanism is relevant to nucleated cells. To illustrate this, the neuroblastoma cell line SHSy5y uses a similar pathway to signal macrophage clearance when undergoing oxidative stress. Thus, inhibition of mannose recognition on these cells may help prevent unwanted cellular clearance, for example, loss of neurons after stroke or in dementia.

### EXAMPLES

The following examples serve to illustrate and support the claimed invention and are not to be construed as limiting in any way. A person who is skilled in the art will appreciate that modifications of the embodiments described herein can be made without departing from the scope of the claimed invention.

Glycomic Analyses

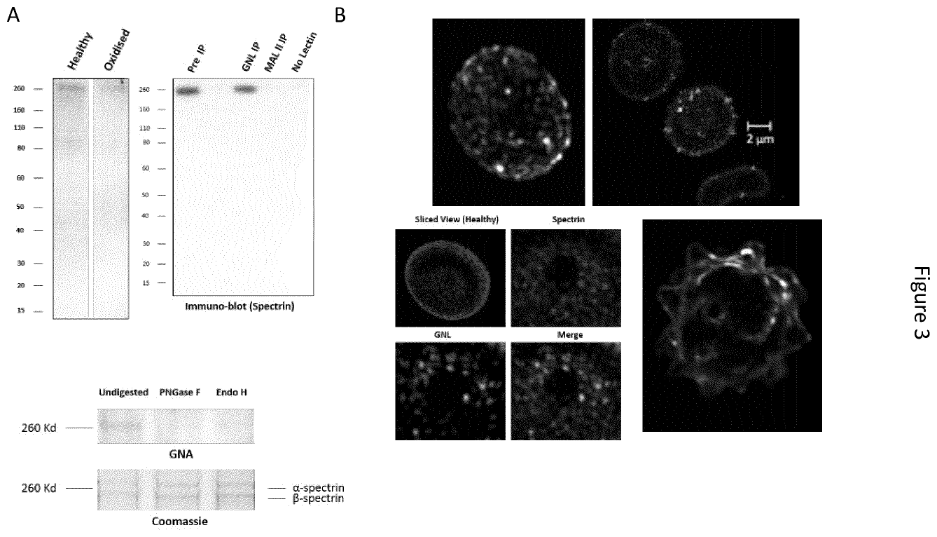

Glycomic analysis was performed and suggested that high mannose species were associated with the RBC plasma membrane. In order to test whether these were available as putative ligands at the cell surface, and whether any such display was dependent on the health of the cell, flow cytometry was used. The surface of untreated and oxidised RBC were probed with panels of lectins, including Galanthus nivalis lectin (GNA) and Narcissus pseudonarcissus lectin (NPL) (FIG. 2). GNA and NPL are of plant origin and bind to terminal mannose. Each of these lectins has a specificity for a different sugar structure.

None of the plant or animal lectins with mannose specificity bound untreated RBC from healthy donors, demonstrating that the high mannose structures identified by glycomic analyses were not displayed on the surface of healthy cells. By contrast, GNA, and the carbohydrate binding domain of the mannose receptor CD206 (MR-CRD), which recognises terminal mannoses, stained oxidised RBC. The specificity of the staining for high mannose was confirmed in both cases by effective blockade with mannan (FIG. 2B), which is a competing mannose polymer, and by the inability of the murine lectins that recognise other sugars, including the CD206 CR domain, to bind the stressed RBC.

Thus, high mannose structures that are cryptic in healthy cells, but exposed after oxidation, were identified.

High Mannose in Healthy Cells

High mannose was visualised in healthy cells using fluorescent microscopy. GNA binding to high mannose on the oxidised RBC was visualised, revealing that the staining was not uniform, but restricted to discrete islands on the cell surface (FIG. 2E, 3B). In line with the results of flow cytometry, there was no GNA staining of untreated RBC. However, when the membranes of these untreated RBC were permeabilised, abundant foci of intracellular staining with GNA was observed, indicating that the high mannose species are cryptic in healthy cells because they are sequestered inside the membrane (FIG. 2E).

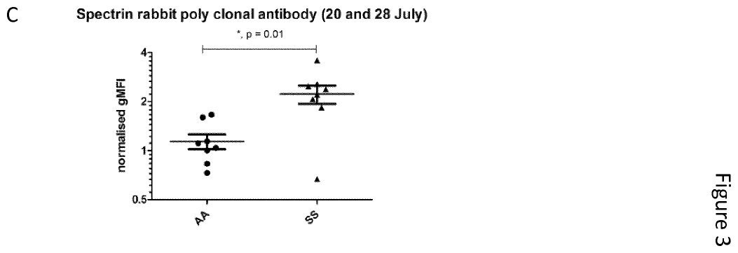

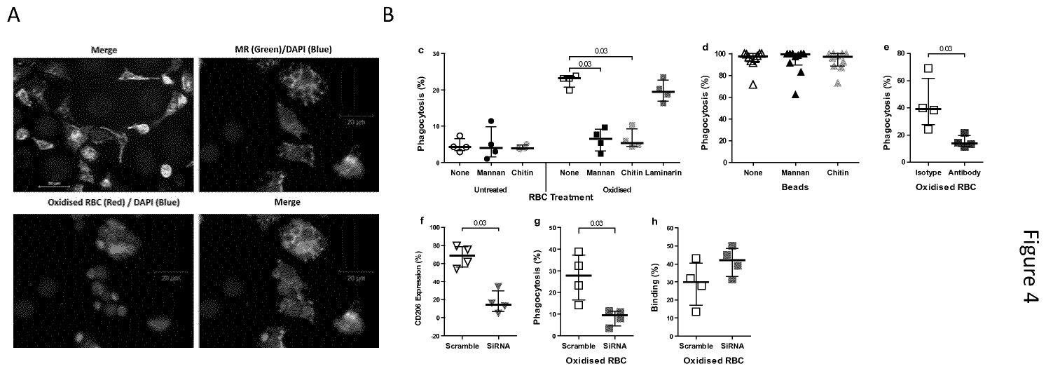

It is unlikely that the exposure of high mannose structures on the surface of oxidised RBC was due simply to loss of membrane integrity, analogous to that seen in necrosis, because this method of stressing RBC is not known to permeabilise cells, and can be used to model the effects of ageing [Burger et al 2014]. Furthermore, that possibility was formally excluded by demonstrating that antibodies that bind epitopes on the cytoplasmic face of integral membrane proteins did not stain the oxidised RBC (FIG. 2D). An antibody specific for O-linked N-acetylglucosamine (O-GlcNAc) also failed to bind the surfaces of oxidatively damaged RBC or those from patients with SCD, demonstrating that the results were not confounded by any ability of GNA to recognise not only mannose, but also O-GlcNAc, which has been reported to decorate a variety of cytoplasmic proteins under conditions of stress [20]. Furthermore, polyclonal antibodies to spectrin were able to bind the surfaces of RBC from patients with SCD, but not those from healthy individuals (FIG. 3C).

Intracellular N-linked high mannose structures outside the Golgi apparatus or endoplasmic reticulum, both of which are absent in mature RBC, have not previously been described. In order to identify which proteins carried these motifs, we fractionated untreated or oxidised RBC ghosts by SDS-PAGE, and probed the corresponding Western blots with GNA (FIG. 1E). In both treated and untreated RBC ghosts, the main signal corresponded to the molecular weight expected for alpha- or beta- spectrin (220-260 kD). In addition, especially in RBC ghosts from patients with SCD, lower molecular weight bands could be detected by lectin blotting with either GNA or daffodil, and these bands were detected by anti-spectrin antibodies, which we interpret as corresponding to spectrin being subjected to proteolytic cleavage.

To confirm that the bands were mannose specific, deglycosylation experiments were performed on the ghosts, which showed that the signal was degraded by both incubation with peptide:N-glycosidase F (PNGase F), indicating an N-linkage, or endoglycosidase H (endoH), indicating high mannose (FIG. 1F). Our interpretation of these results, that spectrin carries N-linked high mannose, was supported by precipitation of solubilised ghost proteins with GNA, followed by Western blotting, which again yielded one band of ˜260 kD that was detected with an antibody specific for alpha-spectrin (FIG. 3A).

Thus, the high mannose structures that are cryptic in healthy RBC are bound to spectrin.

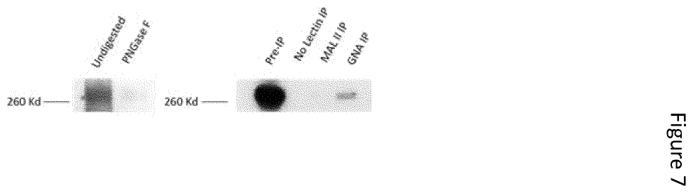

Spectrin is the major component of the membrane skeleton that is important for maintaining the morphology and structural integrity of RBC [22]. Non-erythroid isoforms of spectrin are also ubiquitous in nucleated cells [22]. To determine whether the decoration of spectrin N-linked high mannose species occurs in other cells, neuroblastoma derived cell line SHSy5y were studied. Neurons prominently express a form of spectrin, SPTBN1, which, as with RBC, is important in morphology [21]. Protein extracts of SHSy5y were fractionated by SDS-PAGE, transferred to Western blots and probed with GNA. One band on the Western blots one band corresponded to the predicted molecular mass of SPTBN1, although multiple other bands were also detected, as expected for a cell actively synthesising proteins in the endoplasmic reticulum and Golgi apparatus. The band that corresponds with the molecular mass of SPTBN1 was abolished by deglycosylation of the protein extract with PNGase F prior to fractionation, demonstrating that the GNA binding was specific to N-linked glycan. Furthermore, precipitation from the protein extract with GNA and subsequent Western blotting identified a single band that stained with anti-SPTBN1 antibody, and which exhibited the predicted migration of SPTBN1 (FIG. 7).

Thus, high mannose structures are also bound to the spectrin protein of some non-RBC, for instance neuronal spectrin.

The effect of oxidative stress on SHSy5y was also investigated. As expected for an intracellular protein, fluorescent microscopic examination of untreated (i.e. healthy) SHSy5y monolayers showed no staining with anti-SPTBN1 antibody unless the cells were permeabilised. However, oxidative damage led to the appearance of discrete islands of anti-SPTBN1 binding on both cell bodies and neurites (FIG. 7), similar to those exposed on stressed RBC.

Thus, both the glycosylation of the membrane skeleton, and its exteriorisation when dying, are not unique to RBC, but are also observed on nucleated cells.

Subcellular Distribution of High Mannose

Structured illumination microscopic (SIM) examination of untreated RBC, following permeabilisation and intracellular staining, revealed multiple foci of GNA staining within the network of spectrin, immediately under the plasma membrane (FIG. 3B). This also indicates that only a subset of spectrin molecules is glycosylated, and these are confined to foci within the membrane skeleton.

High Mannose as a Marker for Efferocytosis

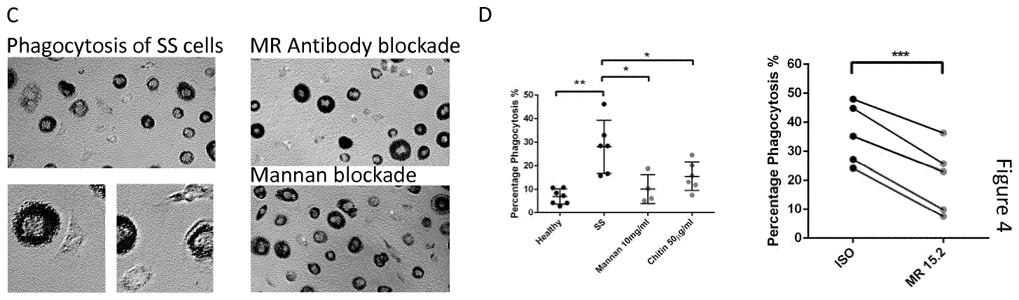

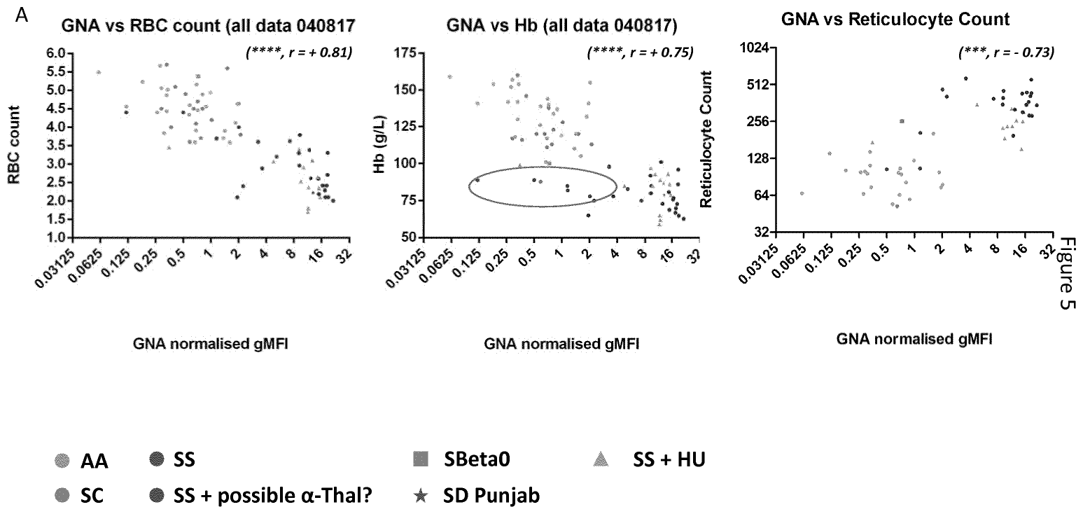

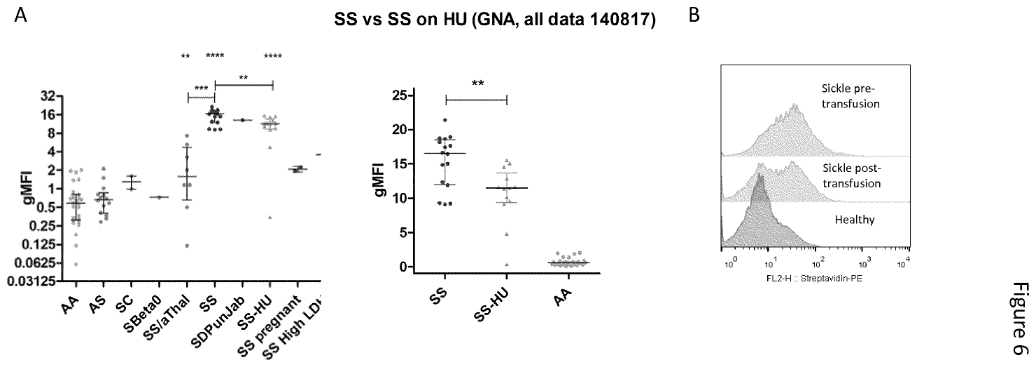

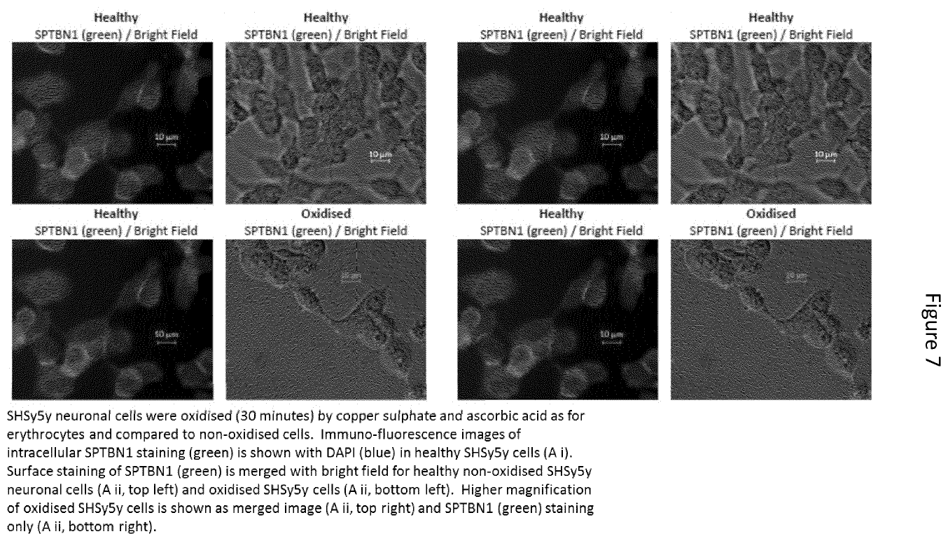

The uptake of oxidised RBC by HMDM in the presence and absence of mannan was compared. Mannan is a linear mannose polysaccharide. The capacity of mannan to compete with high mannose species for any receptor binding was observed (FIG. 4B, 4D). Mannan significantly blocked uptake, and this reduction was specific to RBC, because the polymer had no effect on phagocytosis of sepharose beads by the HMDM. This indicates that efferocytosis involves mannose binding.

The Role of CD206

It was determined whether uptake of oxidised RBC was associated with CD206 expression on HMDM. Immunofluorescence microscopy revealed that CD206 levels vary between macrophages, and that uptake of RBC positively correlates with CD206 expression.

In addition to high mannose species CD206, is known to recognise fungal chitin, but not laminarin. The capacity for these sugar polymers to block RBC efferocytosis was measured (FIG. 2c). Both mannan and chitin, but not laminarin, significantly inhibited uptake of oxidised, but not untreated, RBC.

The role of CD206 in the uptake of oxidised RBC was confirmed by showing that uptake can be blocked by a specific anti-CD206 antibody (FIG. 2B) and can be blocked by siRNA mediated CD206 knockdown (FIG. 2B). These CD206 inhibitors significantly to reduce efferocytosis of oxidised RBC.

The role of CD206 in efferocytosis is surprising because CD206 has often been considered an endocytic, rather than a phagocytic, receptor [14], but our findings are in line with reports that its expression is the best marker distinguishing phagocytic from non-phagocytic macrophages in vivo [1].

Notwithstanding the importance of CD206 for mannose recognition and efferocytosis, the involvement of other receptors is likely, since CD206 knock-down did not prevent binding of oxidised RBC to HMDM and c-type lectin expression is known to vary between different phagocyte populations.

Verification in Disease Samples

To investigate whether inappropriate mannose expression can be pathogenic, the inventors studied RBC from sickle cell disease (SCD) patients. SCD is characterised by an incompletely understood, accelerated clearance of RBC that is associated with oxidative stress of the cells. SCD results in abnormal haemoglobin, termed haemoglobin S.

RBC from patients homozygous for haemoglobin S, probed for surface mannose by GNA, exhibited remarkably high staining in flow cytometric analyses (FIG. 2B). Furthermore, microscopy showed that a high proportion of RBC from patients with SCD displayed discrete surface patches that bound GNA (FIG. 2E), similar to those seen on the deliberately oxidised RBC from healthy donors.

In functional experiments, RBC from patients with SCD were readily taken up by HMDM (FIG. 3e), and preferentially by those macrophages expressing CD206. This uptake was significantly inhibited by competition from mannan and chitin (FIG. 2D) and this finding is consistent with exposure of mannose on the RBC driving efferocytosis. The inventors conclude the mechanism we describe is important in mediating the haemolysis of SCD.

Conclusions

The data disclosed herein indicates that cellular distress causes surface exposure of a previously undisclosed class of molecules, membrane skeleton proteins decorated with high mannose structures that act as markers for efferocytosis [12]. The concentration of exposed mannose into discrete patches may contribute to efficient signalling for uptake by phagocytes.

Moreover, the involvement of spectrins in this mechanism is noteworthy. Spectrins are located just under the plasma membrane in virtually all mammalian cells. Spectrins play important roles in maintaining membrane integrity and cellular shape, are involved in the organization of specialised membrane domains [2] and they may provide a linked series of anchor points for uptake of entire cells.

Exposure of other, more mobile, cryptic signals for phagocytosis on distressed cells, including PS and, for RBC, senescent cell antigen, may alone be less efficient in mediating such clearance of entire cells. This work also sheds light on the parallel evolution of receptors for tissue homeostasis and protective immunity, since CD206 as a representative of the C-type lectin receptor family is here identified as responsible for the recognition of cellular distress, in addition to its previously defined roles as an innate receptor for microbial.

The present disclosure demonstrates that the exposure of spectrin bearing cryptic N-linked mannose signals can drive pathology in SCD, providing a novel target for therapy of haemolytic anaemias.

Materials & Methods

Donors and Consent

Healthy donors and sickle cell disease patients homozygous for the sickle haemoglobin were consented before blood donation. Ethical approval was given for study, Immunomodulatory properties of Red Blood Cells', North of Scotland REC Number 11/NS/0026.

RBC Isolation

Whole blood was collected into vacutainers containing an acid citrate dextrose solution (ACD; Grenier). RBC were isolated by density centrifugation using metrizoate solution in sterile conditions (Lymphoprep; Axis-Shield) (Stott, Barker Urbaniak 2000). Packed RBC were diluted with equal volume of DMEM (4.5 g/L glucose, L-glutamine; Gibco) and stored in ACD solution in sterile conditions (9 ml RBC/DMEM per ACD tube). RBC were stored at 4° C. and used within three days.

Human Monocyte Derived Macrophage Culture

Mononuclear cells were isolated by density centrifugation in conjunction with RBC in sterile conditions (Stott, Barker Urbaniak 2000). Mononuclear cells were seeded at 106 cells/ml in RPMI, 100 U/ml penicillin, 100 μg/ml streptomycin, 292 μg/ml L-glutamine (Gibco) and 10% heat inactivated autologous serum, cells were then incubated at 37° C. with 5% CO2 for 14-21 days. Cells were then washed prior to assays.

Two morphologically distinct subsets of macrophages were observed in the HMDM both demonstrate ability to phagocytose necrotic nucleated cells and latex beads (data not shown): 1) large and granular and 2) small and non-granular, often having characteristic spindle shaped morphology. Erythrocyte binding and phagocytosis is consistently associated with the small and non-granular subpopulation of macrophages and quantification of phagocytosis and binding of erythrocytes is restricted to this subset of macrophages in this study.

SHSy5y Neuronal Cell Culture

SHSy5y (Sigma Aldrich) cell line was cultured according to ATCC recommendations. DMEM-F12 is used as culture medium. (www.atcc.org/˜/ps.CRL-2266.ashx) Mycoplasma testing not completed.

RBC and Neuronal Cell Oxidation

RBC and SHSy5y neuronal cells were incubated with copper sulphate (CuSO4, 0.2 mM) and ascorbic acid (5 mM) for 60 minutes (RBC) or 30 minutes (SHSy5y) at 37° C. in DMEM with 4.5 g/L glucose. Cells were then washed in PBS three times prior to assays.

Eryptotic RBC Damage

RBC eryptosis was induced by incubation with calcium ionophore (2 μM; Sigma Aldrich A23187) for three hours at 37° C. in DMEM with 4.5 g/L glucose.

Efferocytosis and Bead Phagocytosis Assays

For clear identification of efferocytosis by microscopy, RBC were stained with cell trace far red (CTFR; Molecular Probes) according to manufacturer's instructions. RBC were added to HMDM at 5×107 cells per well for three hours before gentle removal and fixation with 4% paraformaldehyde. To identify cells bound but not and ingested by HMDM the cells were stained with anti-glycophorin A/B FITC (HIR2, Biolegend).

Coumarin stained Fluoresbrite microparticles (8 μm; Polysciences, Inc.) were added to HMDM at 5×107 beads per well for three hours before gentle removal and suspension in PBS for immediate imaging.

Ligand binding of CD206 was blocked with antibody clone 15.2 (10 μg/ml; BioLegend) by adding antibody or isotype control (10 μg/ml mouse IgG1 kappa clone 107.3, BD Biosciences) for 60 minutes prior to three hour efferocytosis assay and were not removed.

Mannan (10 mg/ml), chitin (50 μg/ml) and laminarin (10 μg/ml; all Sigma Aldrich) were applied for 60 minutes prior to three hour efferocytosis/phagocytosis assay and were not removed.

Cells were imaged at 32 times magnification using Immuno-Fluorescent Microscope (Zeiss).

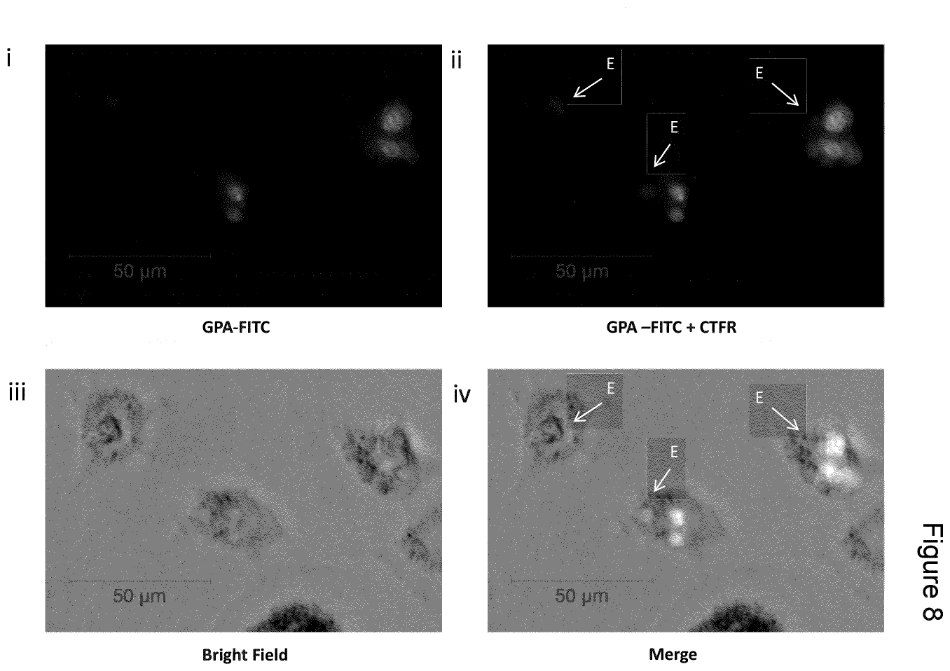

An efferocytic macrophage is defined as exhibiting at least one GPA-FITC negative but CTFR single positive erythrocyte that lies within the boundary of the macrophage in bright field (FIG. 8).

Reactive Oxygen Species Formation

The rate of total ROS formation was determined by loading RBC with oxidation sensitive dye CM-H2DCFDA (10 μM; Molecular Probes) in PBS and incubating for 60 minutes in the dark at 37° C. RBC were washed three times and resuspended in DMEM and fluorescence determined immediately by spectrofluorimeter (Fluostar optima; BMG Labtech). The rate of formation of the fluorescent derivative, was proportional to the intracellular radical production at 37° C. over six hours at an excitation of 485 nm and emission 530 nm.

Flow Cytometry

To analyse phosphatidylserine exposure on RBC, FITC conjugated annexin V (Biolegend) was incubated with in calcium buffer (10 mM HEPES, 2.5 mM CaCl2.H2O, 150 mM NaCl, pH 7.4) for 30 min at room temperature. Cells were then washed and analysed.

RBC, approximately 5×10⁶ per test, were washed three times in PBS, and then incubated in calcium buffer with biotinylated GNA (4 μg/ml, Vector Laboratories, B1245) or in PBS for PNA-FITC (2 μg/ml Sigma Aldrich L7381) for 30 minutes in calcium buffer at room temperature (protected from light). Cells were then washed and incubated with streptavidin PE-Cy7 (0.27 μg/ml; eBioscience) or PE (0.67 μg/ml; BD Pharmingen) for 30 min at room temperature. Cells were washed and analysed.

Humanised FC fusions of murine C-type lectins (5 μg/ml, kind gift from Gordon Brown, Screening for Ligands of C-Type Lectin-Like Receptors, Elwira PyżGordon D. Brown, 2011) were incubated with RBC for 30 minutes at room temperature in calcium buffer then detected by Alexa Fluor 647 goat anti-human secondary antibody (2 μg/ml, 109-605-098, Jackson ImmunoResearch Laboratories) incubated for 30 minutes at room temperature.

For blockade testing, Lectin or FC fusion were pre-incubated with mannan (5 mg/ml, unless otherwise stated) for 15 minutes at room temperature. Mixture of mannan and Lectin or FC fusion was then incubated with washed RBC and compared to binding of Lectin or FC fusion without mannan.

Samples were acquired on FACSCalibur (BD) and analysed using FlowJo v10.0 (Treestar) software. The normalised geomean was calculated by subtracting the geomean of the secondary only paired controls. For PNA-FITC analysis, normalised geomean was calculated by subtracting geomean of unstained RBC (PBS incubation) control.

Biotinylated BRIC-132 (10 μg/ml 9458B, IBGRL), Biotinylated BRIC-163 (10 μg/ml 9410B, IBGRL), O-GlcNAc (RL2 1 μg/ml, 59624 Santa Cruz) binding was performed (PBS, 30 minutes, room temperature). Streptavidin secondary (BRIC-132/163) and anti-mouse PE secondary (O-GlcNAc) were applied (PBS, 30 minutes, room temperature)

Permeabilisation

Glutaldehyde fixed (0.005%, 10 minutes, room temperature) RBC were permeabilised with Triton X-100 (0.1%, freshly made in PBS, 5 minutes, room temperature). Permabilised RBC were washed in PBS.

Immuno-Fluorescent Microscopy:

Erythrocyte Imaging

Erythrocytes were imaged at 32 times magnification using Immuno-Fluorescent Microscope (Zeiss). Cell surface GNA binding experiment was prepared as for flow cytometry with minor alterations (107 cells per test, 8 μg/ml GNA, lug/ml Streptavidin PE). Intracellular GNA binding was performed following fixation (0.005% glutaraldehyde/PBS, 10 minutes, room temperature) and permeabilisation (0.1% TritonTM-X 100/PBS, 15 minutes, room temperature). Stained cells were pulse centrifuged for 30 seconds (less than 1800 rpm RCF including acceleration in 24 well, flat bottom tissue culture plates (Greiner) before imaging. Images analysed by Zen (Black and Blue versions, Zeiss).

Qualitative macrophage efferocytosis imaging with mannose receptor was performed post efferocytosis assay and fixation. Washed cells were blocked for 15 minutes in 1% BSA/PBS at room temperature in dark and stained with Alexa-488 conjugated mannose receptor antibody (1.25 μg/ml, Clone 19.2, 53-2069-47, eBiosciences) and DAPI (D1306, Thermo Fisher, used as per manufacturer's instructions) for 30 minutes at room temperature. Cells were washed in PBS prior to microscopy.

Confocal Microscopy

For spectrin-GNA double staining experiments, permeabilised (see Immuno-Fluorescent Micoscopy section) erythrocytes were stained with anti-human spectrin antibody (Sigma, S3396, 1 in 50 dilution, Manufacturer's stock concentration unknown) concurrently with GNA (8 μg/ml) in calcium buffer. Alexa Fluor 647 anti-mouse antibody (10 μg/ml, Thermo Fischer, double check) was applied in conjunction with streptavidin PE (1 ug/ml, Beckman Dickinson) following staining of primary reagents. RBC was gravity sendimented (30 minutes room temperature, in dark) on to poly-L-lysine (Sigma Aldrich) treated 8 well chamber slides (LabTek). Confocal microscopy was performed using a Zeiss LSM 710.

Transmission Electron Microscopy

Oxidised erythrocytes were fixed with 2.5% Glutaraldehyde in 0.1M Sodium cacodylate buffer pH 7.4 for 4 hrs and then post fixed in 1% Osmium Tetroxide in distilled water for 1 hr, then dehydrated in ethanol and infiltrated and embedded in Spurrs resin. Ultrathin 70 nm sections were prepared and stained with uranyl acetate and lead citrate, before being viewed with a JEOL 1400 plus transmission electron microscope at 80 kV.

3D-Structured Illumination Microscopy

RBC were stained as per confocal microscopy and gravity sedimented into poly-L-lysine treated chamber slide (LabTek). Images were rendered and processed in Imaris (Bitplane).

Erythrocyte Ghost Preparation

Membrane ghost preparation from healthy and oxidised erythrocytes was adapted from (Barker et al 1991, Barker et al 1992) Washed erythrocytes were subjected to hypotonic lysis (20 mM Tris, pH 7.6, ice cold, protease inhibitor, Pierce, double check) on ice. Lysates were washed three times in hypotonic lysis buffer (37044 g, 4° C., 30 minutes, no brake). Washed erythrocyte ghost was resuspended in minimal hypotonic lysis buffer for analysis.

Immuno-Blotting

Erythrocyte ghost protein concentration was determined by protein BCA assay (Pierce, double check). Ghost preparation was mixed in equal volume with 8M urea sample buffer (Barker et al 1991, Barker et al 1992) and denatured by heating at 100° C. for 10 minutes. Ghost protein samples were separated by gel electrophoresis (Novex, 4-12% Bis Tris gel, MOPS buffer) and subjected to immuno-blotting with biotinylated GNA lectin (40 μg/ml, Vector Laboratories) and streptavidin HRP (Cell Signalling). Ghost protein loading was normalised by protein concentration (approximately 6 ug per sample).

Despite manufacturer's claim of binding to both alpha and beta spectrin, a single band corresponding to the size of alpha spectrin is only detected by immune-blotting using Sigma, S3396.

Colloidal Coomassie Staining and Mass Spectrometry

Ghost protein samples separated by electrophoresis as described above were subjected to colloidal Coomassie staining. Bands corresponding to putative alpha and beta spectrin were excised and subjected to trypsin digest and mass spectrometry (Stuart to add/correct details here)

Immuno Precipitation

Erythrocyte ghost was treated with equal volume of binding buffer containing 2% Triton-X 100. Triton treated erythrocyte ghost was pre-cleared with magnetic streptavidin beads (Pierce) and incubated with biotinylated GNA lectin (Vector Laboratories), biotinylated MAL-II lectin (Vector Laboratories) or no lectins overnight at 4° C. in binding buffer. Magnetic precipitation with magnetic streptavidin beads was performed in binding buffer and magnetic beads were washed with binding buffer containing 0.1% Triton-X 100. Washed precipitates were denatured at 100° C. for 10 minutes and supernatants were loaded for immuno-blotting.

siRNA

To access percentage MR positive spindle/small round macrophages, anti-MR antibody (unlabelled clone 19.2) was stained with anti-mouse PE secondary antibody. Only spindle/small round macrophage population was examined as this is the subpopulation that expresses MR in wild type macrophages. SiRNA (UACUGUCGCAGGUAUCAUCCA, antisense, concentration Life Technologies) against mannose receptor (MRC-1) in humans was transfected into primary macrophages (RNAiMax, Life Technologies). (n=4 donors for all SiRNA experiments. Autologous red blood cells were used with macrophages.) Mannose receptor expression was established by microscopy using MR-Alexa-488 staining (described above) in the small non-granular macrophage sub-population by merging bright field and mannose receptor fluorescence staining.

Statistics

All data has been treated as non-parametric and presented (where appropriate) with median and interquartile range. Statistical significance was assessed by either two-tailed Mann-Whitney (non-paired data) and two-tailed Wilcoxon signed rank tests (paired data).

### REFERENCES

1. A-Gonzalez, N. et al. Phagocytosis imprints heterogeneity in tissue-resident macrophages. J. Exp. Med. 214, 1281-1296 (2017).

2. Baines, A. J. The spectrin-ankyrin-4.1-adducin membrane skeleton: adapting eukaryotic cells to the demands of animal life. Protoplasma 244, 99-131 (2010).

3. Bilyy, R. O. et al. Macrophages discriminate glycosylation patterns of apoptotic cell-derived microparticles. J. Biol. Chem. 287, 496-503 (2012).

4. Boguslawska, D. M., Machnicka, B., Hryniewicz-Jankowska, A. & Czogalla, A. Spectrin and phospholipids—the current picture of their fascinating interplay. Cell. Mol. Biol. Lett. 19, 158-179 (2014).

5. Dini, L., Pagliara, P. & Carla, E. C. Phagocytosis of apoptotic cells by liver: a morphological study. Microsc. Res. Tech. 57, 530-540 (2002).

6. Duvall, E., Wyllie, A. H. & Morris, R. G. Macrophage recognition of cells undergoing programmed cell death (apoptosis). Immunology 56, 351-358 (1985).

7. Erwig, L. P. & Gow, N. A. Interactions of fungal pathogens with phagocytes. Nat. Rev. Microbiol. 14, 163-176 (2016).

8. Fadok, V. A. et al. Exposure of phosphatidylserine on the surface of apoptotic lymphocytes triggers specific recognition and removal by macrophages. J. Immunol. 148, 2207-2216 (1992).

9. Falasca, L., Bergamini, A., Serafino, A., Balabaud, C. & Dini, L. Human Kupffer cell recognition and phagocytosis of apoptotic peripheral blood lymphocytes. Exp. Cell Res. 224, 152-162 (1996).

10. Gardai, S. J., Bratton, D. L., Ogden, C. A. & Henson, P. M. Recognition ligands on apoptotic cells: a perspective. J. Leukoc. Biol. 79, 896-903 (2006).

11. Gardai, S. J. et al. Cell-surface calreticulin initiates clearance of viable or apoptotic cells through trans-activation of LRP on the phagocyte. Cell 123, 321-334 (2005).

12. Hart, G. & West, C. in Essentails of Glycobiology, 2nd edition (ed Ajit Varki, Richard D Cummings, Jeffery D Esko, Hudson H Freeze, Pamela Stanley, Carolyn R Bertozzi, Gerald W Hart, and Marilynn E Etzler) Chapter 17 (Cold Spring Harbor Laboratory Press, Cold Spring Harbor (NY), 2009).

13. Kiyotake, R. et al. Human Mincle Binds to Cholesterol Crystals and Triggers Innate Immune Responses. J. Biol. Chem. 290, 25322-25332 (2015).

1. Lutz, H. U. & Bogdanova, A. Mechanisms tagging senescent red blood cells for clearance in healthy humans. Front. Physiol. 4, 387 (2013).

14. Martinez-Pomares, L. The mannose receptor. J. Leukoc. Biol. 92, 1177-1186 (2012).

15. Piel, F. B., Steinberg, M. H. & Rees, D. C. Sickle Cell Disease. N. Engl. J. Med. 376, 1561-1573 (2017).

16. Poon, I. K., Lucas, C. D., Rossi, A. G. & Ravichandran, K. S. Apoptotic cell clearance: basic biology and therapeutic potential. Nat. Rev. Immunol. 14, 166-180 (2014).

17. Pretorius, E., du Plooy, J. N. & Bester, J. A Comprehensive Review on Eryptosis. Cell. Physiol. Biochem. 39, 1977-2000 (2016).

18. Pyz, E. & Brown, G. D. Screening for ligands of C-type lectin-like receptors. Methods Mol. Biol. 748, 1-19 (2011).

19. Wagener, J. et al. Fungal chitin dampens inflammation through IL-10 induction mediated by NOD2 and TLR9 activation. PLoS Pathog. 10, e1004050 (2014).

20. Wang, Z. et al. Site-specific GlcNAcylation of human erythrocyte proteins: potential biomarker(s) for diabetes. Diabetes 58, 309-317 (2009).

21. Xu, K., Zhong, G. & Zhuang, X. Actin, spectrin, and associated proteins form a periodic membrane skeletal structure in axons. Science 339, 452-456 (2013).

22. Zhang, R., Zhang, C., Zhao, Q. & Li, D. Spectrin: structure, function and disease. Sci. China Life. Sci. 56, 1076-1085 (2013).

Freeman S A, Grinstein S. Phagocytosis: receptors, signal integration, and the cytoskeleton. Immunol Rev. 2014 November; 262(1):193-215. doi: 10.1111/imr.12212.

J Clin Invest. 2017 Mar. 1; 127(3):750-760. doi: 10.1172/JCI89741. Epub 2017 Mar. 1.

Intravascular hemolysis and the pathophysiology of sickle cell disease.

Kato G J, Steinberg M H, Gladwin M T.

Identification of alloreactive T-cell epitopes on the Rhesus D protein

Lisa-Marie Stott, Robert N. Barker and Stanislaw J. Urbaniak Blood 2000 96:4011-4019;

Screening for Ligands of C-Type Lectin-Like Receptors, Elwira PyżGordon D. Brown, 2011

CD47 functions as a molecular switch for erythrocyte phagocytosis.

Burger P, Hilarius-Stokman P, de Korte D, van den Berg TK, van Bruggen R. Blood. 2012 Jun. 7; 119(23):5512-21. doi: 10.1182/blood-2011-10-386805.

1. (canceled)

2. A method of treating a haemolytic anaemia in a mammalian subject, the method comprising administering
an inhibitor of the interaction between an innate immune receptor and glycans expressed on the surfaces of diseased red blood cells to the subject.

3. The method of claim 2, wherein the innate immune receptor is a C-type lectin.

4. The method of claim 3, wherein the C-type lectin is the mannose receptor (CD206).

5. The method of claim 2, wherein the inhibitor is a decoy ligand that binds to the innate immune receptor.

6. The method of claim 5, wherein the decoy ligand comprises an oligosaccharide or polysaccharide.

7. The method of claim 6, wherein the oligosaccharide or polysaccharide comprises mannose or an analogue thereof.

8-10. (canceled)

11. The method of claim 4, wherein the inhibitor is an anti-CD206 antibody.

12. The method of claim 4, wherein the inhibitor is a nucleic acid that causes CD206 knock-down or CD209 knock-down.

13. The method of claim 2, wherein the inhibitor is a decoy ligand that binds to the glycan expressed on the surface of damaged cells.

14. The method of claim 13, wherein the damaged cells are damaged red blood cells.

15. (canceled)

16. The method of claim 13, wherein the agent binds high mannoses.

17. The method of claim 16, wherein the decoy ligand comprises an agent that binds high mannoses expressed on damaged or diseased red blood cells.

18. The method of claim 15, wherein the decoy ligand is a decoy receptor comprising the CRD of CD206.

19. The method of claim 2, wherein the therapeutic agent comprises a pharmaceutically acceptable excipient, carrier, buffer and/or stabiliser.

20. The method of claim 2, wherein the mammal is a human.

21-33. (canceled)

34. A method comprising detecting the presence or absence of high mannose glycans on the surface of red blood cells in a sample that has been obtained from a subject who is suspected of having an anaemia.

35. The method of claim 34, wherein the method comprises contacting a blood sample with a fluorescently labelled lectin.

36. The method of claim 35, wherein the method comprises using flow cytometry to detect the fluorescently labelled lectin bound to the high mannose glycans on the surface of red blood cells in the sample.

37. A method of detecting damaged or diseased red blood cells, the method comprising detecting high mannose structures on the surface of the red blood cells.

38. The method of claim 37, wherein the method comprises contacting a blood sample with a fluorescently labelled lectin.

39. The method of claim 38, wherein the method comprises using flow cytometry to detect the fluorescently labelled lectin bound to the high mannose glycans on the surface of red blood cells in the sample.
